(19) United States
(12) Patent Application Publication
(10) Pub. No.: US 2015/0320311 A1
(43) Pub. Date: Nov. 12, 2015

(54) METHOD AND APPARATUS FOR IRIS RECOGNITION USING NATURAL LIGHT

(71) Applicant: Here Global B.V., Veldhoven (NL)

(72) Inventors: Xin Chen, Evanston, IL (US); Xinyu Huang, Cary, NC (US)

(21) Appl. No.: 14/275,301

(22) Filed: May 12, 2014

(51) Int. Cl. A61B 3/12; A61B 3/14

(52) U.S. Cl. CPC A61B 3/1216; A61B 3/14

(57) ABSTRACT

A method, apparatus and computer program product are provided for iris recognition using natural light. A method is provided for receiving, at a user device, a plurality of iris images. The iris images are captured using natural light. The method also includes generating a composite iris ring based on the plurality of iris images, receiving an iris pattern, comparing the composite iris ring to the iris pattern, and determining a match probability based on the comparison of the composite iris ring to the iris pattern.

An example embodiment of the present invention relates to biometric data and, more particularly, to iris recognition using natural light.

### BACKGROUND

Some iris recognition systems utilize infrared or near infrared light emission and an infrared or near infrared camera to capture iris images. Infrared light is used to prevent occlusion of the iris pattern, from reflections, glare, or the like caused by natural or visible light.

Due to power constraints and portability requirements of mobile or wearable devices it may be difficult to implement an infrared iris recognition system on such devices.

### BRIEF SUMMARY

A method and apparatus are provided in accordance with an example embodiment for iris recognition using natural light. In an example embodiment, a method is provided that includes receiving, at a user device, a plurality of iris images. The iris images are captured using natural light. The method also includes generating a composite iris ring based on the plurality of iris images, receiving an iris pattern, comparing the composite iris ring to the iris pattern, and determining a match probability based on the comparison of the composite iris ring to the iris pattern.

In an example embodiment, the method also includes determining an iris boundary and a pupil boundary defining an iris ring in the respective iris images of the plurality of iris images, and segmenting the iris ring from the respective iris images of the plurality of iris images. In a further example embodiment, the method also includes extracting unclear regions from the iris ring. The generating a composite iris ring is further based on clear regions of the iris ring.

The method, of an example embodiment, also includes matching iris pattern and iris ring resolution, wherein the comparison of the iris ring and iris pattern is further based on the iris ring and iris pattern having a matched resolution. In some example embodiments the method also includes determining an exposure period based on available natural light and capturing a plurality of iris images. The capture of the plurality of iris images is performed using the determined exposure period.

In an example embodiment the method also includes determining if the match probability satisfies a predetermined match threshold. In some embodiments, the method also includes segmenting the iris ring into iris ring segments.

In another example embodiment an apparatus is provided including at least one processor and at least one memory including computer program code, the at least one memory and computer program code configured to, with the processor, cause the apparatus to at least receive a plurality of iris images. The iris images are captured using natural light. The at least one memory and computer program code are configured to generate a composite iris ring based on the plurality of iris images, receive an iris pattern, compare the composite iris ring to the iris pattern, and determine a match probability based on the comparison of the composite iris ring to the iris pattern.

In an example embodiment of the apparatus, the at least one memory and the computer program code are further configured to determine an iris boundary and a pupil boundary defining an iris ring in the respective iris images of the plurality of iris images, and segment the iris ring from the respective iris images of the plurality of iris images. In some example embodiments of the apparatus, the at least one memory and the computer program code are further configured to: extract unclear regions from the iris ring. The generating a composite iris ring is further based on clear regions of the iris ring.

The at least one memory and the computer program code, of an example embodiment of the apparatus, are further configured to match iris pattern and iris ring resolution. The comparison of the iris ring and iris pattern is further based on the iris ring and iris pattern having a matched resolution. In an example embodiment of the apparatus, the at least one memory and the computer program code are further configured to determine an exposure period based on available natural light, and capture a plurality of iris images. The capture of the plurality of iris images is performed using the determined exposure period.

In some example embodiments of the apparatus, the at least one memory and the computer program code are further configured to determine if the match probability satisfies a predetermined match threshold. The at least one memory and the computer program code, of an example embodiment of the apparatus, are further configured to segment the iris ring into iris ring segments.

In still a further embodiment, a computer program product is provided including at least one non-transitory computer-readable storage medium having computer-executable program portions stored therein, the computer-executable program code portions comprising program code instructions configured to receive a plurality of iris images. The iris images are captured using natural light. The computer-executable program code portions of the computer program device also include program code instructions configured to generate a composite iris ring based on the plurality of iris images, receive an iris pattern, compare the composite iris ring to the iris pattern, and determine a match probability based on the comparison of the composite iris ring to the iris pattern.

In the computer program product of an example embodiment, the computer-executable program code portions further comprise program code instructions configured to determine an iris boundary and a pupil boundary defining an iris ring in the respective iris images of the plurality of iris images and segment the iris ring from the respective iris images of the plurality of iris images. In an example embodiment of the computer program product the computer-executable program code portions further comprise program code instructions configured to segment the iris ring into iris ring segments and extract unclear regions from the iris ring. The unclear regions are unclear iris ring segments, and the generating a composite iris ring is further based on clear regions of the iris ring.

In an example embodiment of the computer program product, the computer-executable program code portions further comprise program code instructions configured to match iris pattern and iris ring resolution. The comparison of the iris ring and iris pattern is further based on the iris ring and iris pattern having a matched resolution. In some embodiments of the computer program product, the computer-executable program code portions further comprise program code instructions configured to determine an exposure period based on available natural light and capture a plurality of iris images The capture of the plurality of iris images is performed using the determined exposure period. In the computer program product of an example embodiment, the computer-executable program code portions further comprise program code instructions configured to determine if the match probability satisfies a predetermined match threshold.

In yet another example embodiment, an apparatus is provided that includes means for receiving a plurality of iris images. The iris images are captured using natural light. The apparatus also includes means for generating a composite iris ring based on the plurality of iris images, means for receiving an iris pattern from a memory, means for comparing the composite iris ring to the iris pattern, and means for determining a match probability based on the comparison of the composite iris ring to the iris pattern.

### BRIEF DESCRIPTION OF THE DRAWINGS

Having thus described example embodiments of the invention in general terms, reference will now be made to the accompanying drawings, which are not necessarily drawn to scale, and wherein:

FIG. 1 illustrates a communications diagram in accordance with an example embodiment of the present invention;

FIG. 2 is a block diagram of an apparatus that may be specifically configured for iris recognition using natural light in accordance with an example embodiment of the present invention;

FIG. 3 illustrates example depictions of iris occlusions in accordance with an embodiment of the present invention;

FIGS. 4 and 5 illustrate example iris images in accordance with an embodiment of the present invention;

FIGS. 6A-6C illustrate example iris images in accordance with an embodiment of the present invention; and

FIG. 7 is a flow chart illustrating the operations performed, such as by the apparatus of FIG. 2, in accordance with an example embodiment of the present invention.

### DETAILED DESCRIPTION

Some embodiments of the present invention will now be described more fully hereinafter with reference to the accompanying drawings, in which some, but not all, embodiments of the invention are shown. Indeed, various embodiments of the invention may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present invention. Thus, use of any such terms should not be taken to limit the spirit and scope of embodiments of the present invention.

Additionally, as used herein, the term ‘circuitry’ refers to (a) hardware-only circuit implementations (for example, implementations in analog circuitry and/or digital circuitry); (b) combinations of circuits and computer program product(s) comprising software and/or firmware instructions stored on one or more computer readable memories that work together to cause an apparatus to perform one or more functions described herein; and (c) circuits, such as, for example, a microprocessor(s) or a portion of a microprocessor(s), that require software or firmware for operation even if the software or firmware is not physically present. This definition of ‘circuitry’ applies to all uses of this term herein, including in any claims. As a further example, as used herein, the term ‘circuitry’ also includes an implementation comprising one or more processors and/or portion(s) thereof and accompanying software and/or firmware. As another example, the term ‘circuitry’ as used herein also includes, for example, a baseband integrated circuit or applications processor integrated circuit for a mobile phone or a similar integrated circuit in a server, a cellular network device, other network device, and/or other computing device.

As defined herein, a “computer-readable storage medium,” which refers to a non-transitory physical storage medium (for example, volatile or non-volatile memory device), can be differentiated from a “computer-readable transmission medium,” which refers to an electromagnetic signal.

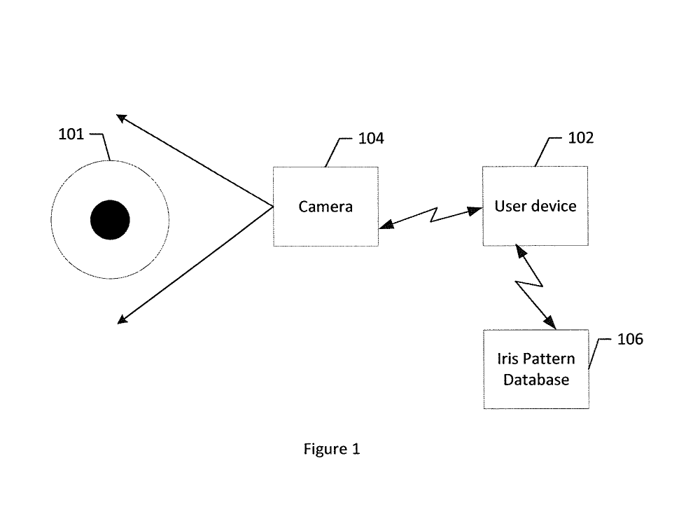

A method, apparatus and computer program product are provided in accordance with an example embodiment for iris recognition using natural light. FIG. 1 illustrates a communication diagram including user equipment (UE) 102, in communications with an iris pattern database 106 and a camera 104. The camera 104 may capture iris images of an eye 101. The UE 102 may be a mobile phone, tablet computer, laptop computer, personal data assistant (PDA), digital television, desktop computer, wearable computing device, or the like. The iris pattern database 106 may be a memory associated with the UE 102, a server, a network, a removable storage, or any other media storage device which is configured for storing one or more iris patterns. The UE 102 may include or be otherwise associated with a camera 104. In example UEs 102, such as a mobile phone, laptop, PDA, or wearable computing device, the camera 104 may be housed as a portion of the UE. Although, application of the invention is described in conjunction with a mobile device, one or ordinary skill in the art would immediately appreciate that it could be embodied in stationary devices, such as a personal computer, computer workstation, a kiosk, or the like.

Various security applications which may be executed on or in association with the UE 102 may require biometric data to access applications, data, or device functions. In an example embodiment, the biometric data may be iris pattern recognition. An iris image pattern, of a user, may be generated and stored in the iris pattern database 106.

The UE 102 may receive an indication that an iris pattern recognition is requested by an application, user, or the like. The UE 102 may cause a sensor, such as a photo sensor, e.g. light sensor, associated with the camera 102 to determine an exposure time based on the available natural light. Available natural light may include sun light or artificial light within the visible light range. Artificial light sources within the visible light range may include incandescent light sources, florescent light sources, light emitting diodes (LED), or the like. In some embodiments, the camera 104 may be equipped or associated with a light source that may be illuminated to increase the natural light available. An exposure time may be the time necessary to capture an image based on the available natural light.

The UE 102 may capture a plurality of iris images using the determined exposure time. The user may be prompted to place their eye 101 near the camera 104. The UE 102 may receive an indication of the eye 101 being positioned near the camera 104. The indication of eye 101 position may be automatically determined by the camera 104 and/or the UE 102 determining that an object or specifically an eye has been positioned near the camera aperture. Additionally or alternatively, the UE 102 may receive an indication of the eye 101 position based on a user input, such as selecting an “in position” or “ready” icon.

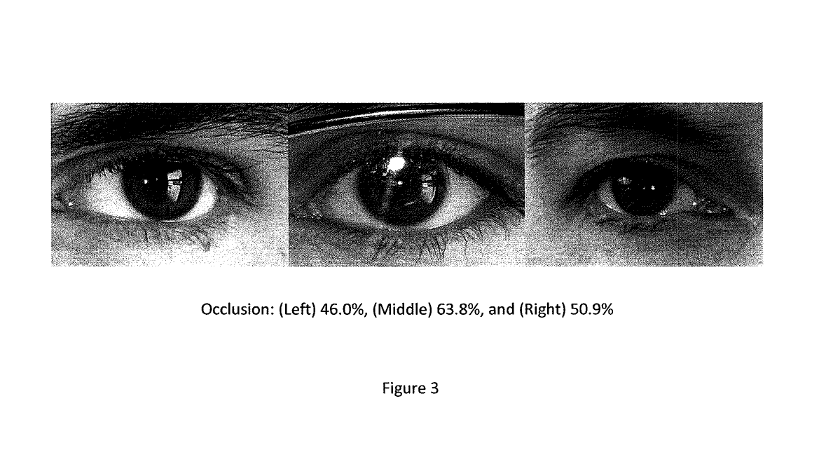

The camera 104 may capture one or more iris images using natural light in a first eye position, such as straight ahead. The iris images may have occasion areas, such as glare, reflection, or blur, from natural light as depicted in FIG. 3. To compensate for occlusion areas, the UE 102 may prompt the user to reposition their eye 101, such as look left, right, up or down, reposition the camera in relationship to the light sources, or the like. The camera 104 may receive an indication of the user's eye 101 being in position and capture one or more additional iris images. In some example embodiments the UE 102 may cause the camera 104 to capture three or more iris image positions. In some embodiments the UE 102 may store the plurality of iris images in a local or remote temporary or long term memory for processing.

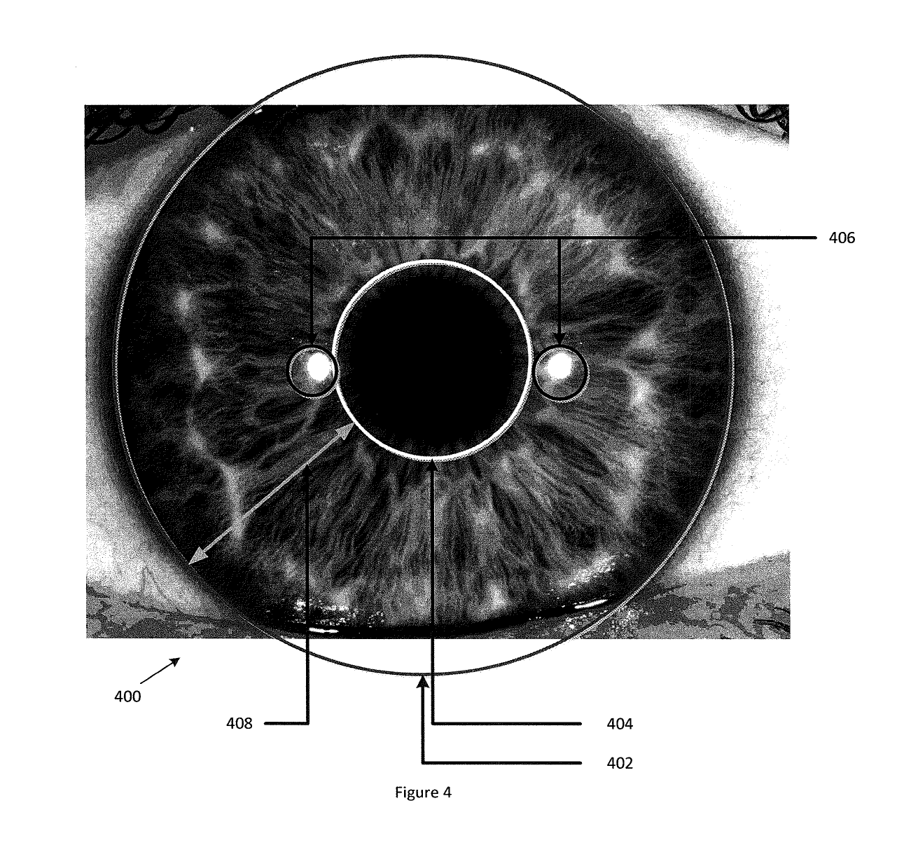

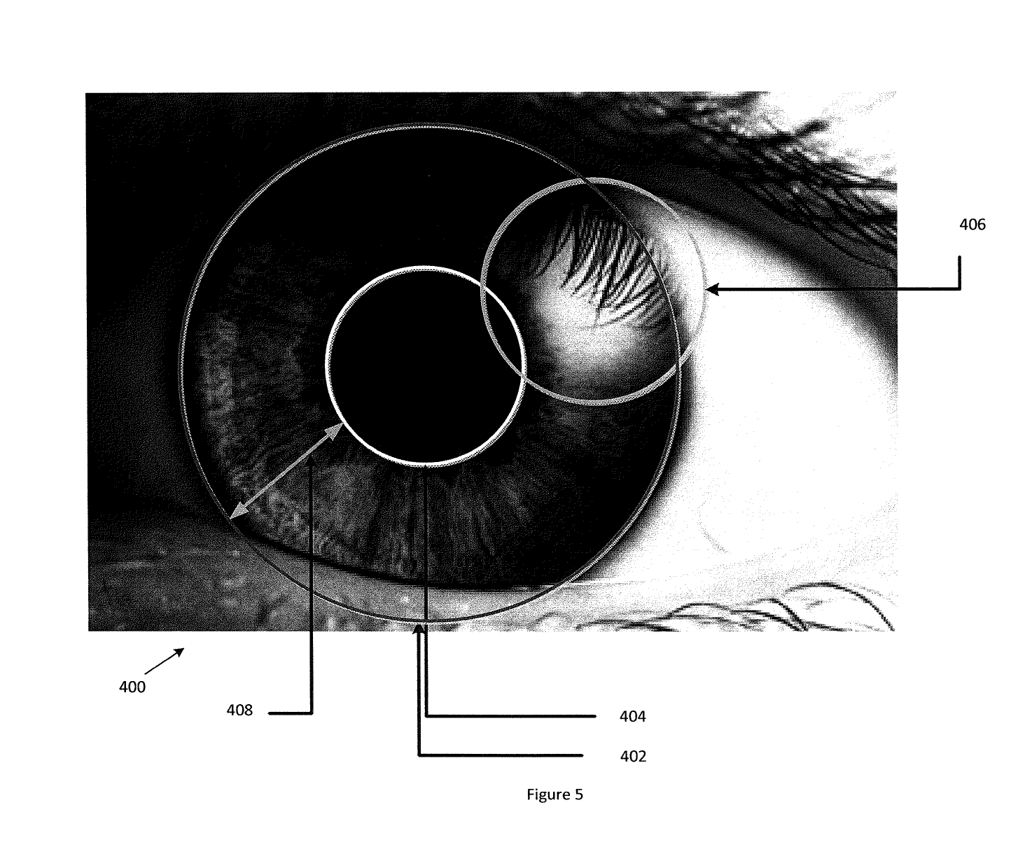

The UE 102 may receive the plurality of iris images from the camera 104 or the memory. The UE 102 may determine the iris and pupil boundaries of each iris image. As depicted in FIGS. 4 and 5, the UE 102 may determine an iris boundary 402, such as a circular boundary at or near the intersection of the iris and the white of the eye in the iris image. The UE 102 may also determine a pupil boundary 404 defining a pupil region. The area between iris boundary 402 and the pupil boundary 404 may be referred to as the iris ring 408. Additionally, the UE 102 may determine unclear regions 408, such as glares, reflections, blurs, or the like, in which the iris pattern of the iris ring is occluded.

The UE 102 may segment the iris ring 408 from each of the plurality of iris images. The UE 102 may remove the iris ring 408 from the iris image 400 and store the iris rings 408 in a temporary or long term memory for processing. In an example embodiment, the UE 102 may further segment the iris rings 408. For example, the full iris ring may be the primary segment and secondary segments may be determined, such as rows, columns, pie pieces, or the like, for individual processing.

In an example embodiment, the UE 102 may extract unclear iris regions from the iris ring. The UE 102 may remove unclear regions 406, e.g. glare, reflections, or blurs, from the iris rings 408. Additionally or alternatively, the UE 102 may remove secondary segments of the iris ring which contain unclear regions 406. After the extraction of the unclear regions 406, clear iris ring portions or segments may remain for each iris ring 408.

In an alternative embodiment, the clear regions of the iris ring or clear iris ring segments may be extracted from the iris ring 408 for processing.

The UE 102 may generate a composite iris ring based on the clear iris ring 408 segments. The UE 102 may identify the overlap of each iris ring 408 in relation to other iris rings in the plurality of iris rings, e.g. iris ring portions or segments which are in multiple iris images. The UE 102 may select the iris ring with the most clear iris segments or the most complete iris ring 408 after the removal of the unclear regions 406. The UE 102 may fill in the missing segments or iris ring regions using clear segments or regions from the other iris image 400 iris rings 408 from the plurality of iris images to generate a composite iris ring in which the whole iris ring is clear, or the maximum available clear iris ring is represented.

In some example embodiments, the UE 102 may determine if the most complete iris ring or the composite iris ring satisfy a predetermined completeness threshold. The completeness threshold may be a minimum percentage of the iris ring that is represented to perform a iris recognition comparison, as discussed below. For example, a completeness threshold may be 80 percent of the iris ring for iris recognition comparison. In an instance in which the completeness threshold is satisfied, the process continues to the iris recognition comparison. In an instance in which the completeness threshold is not satisfied the process may recommence by determining the exposure period and capturing a second plurality of iris images.

In further example embodiments the composite iris ring may be generated by averaging the clear iris ring region or segments. The clear iris ring segments may be aligned, e.g. portions of each iris image iris ring may be matched with portions of other iris image iris rings and associated. The UE 102 may then average the clear iris ring segments or regions, such as by using Gaussian Mixed Models (GMM), or other image averaging model.

The UE 102 may receive an iris pattern from memory, such as a local memory associated with the UE, a server, or the like. The UE 102 may modify the iris pattern to match the resolution of the iris images. The UE 102 may adjust the resolution by increasing or decreasing the area per pixel resolution of the iris pattern, to match the area per pixel resolution of the composite iris ring or most complete iris ring.

The UE 102 may compare the most complete iris ring or the composite iris ring to the iris pattern received from memory. The UE 102 may identify iris pattern identification points associated with the iris ring, such as rings, furrows, freckles, or the like, and compare the iris ring identification points to the identification points of the iris pattern received from memory.

Additionally or alternatively, the UE 102 may determine a match be determining an iris ring variance, e.g. uncertainty, for each area of the iris region. An area of the iris region may be the entire iris ring iris ring subsections, iris ring segments, individual pixels, or any other sub division of the iris ring. The UE 102 may assign a variance weight to each iris ring area, the weight may be scaled or binary, such as a 1-5, 5 being no variation and 1 being highest variation, or in binary 1 being no or little variation, e.g. match and 0 being variation satisfying a variation threshold, e.g. no match. The UE 102 may determine the average weight of the iris ring areas.

The UE 102 may determine a match probability based on the number of identification points which are determined to be a match based on the comparison of the iris ring to the iris pattern and/or the average weight of the iris ring variance. The match probability may be a percentage of matching identification points or a function of the average weight of the iris variance. For example, in an instance in which the variation is scaled 1-5, 5 being no variation and 1 being highest variation, the UE may determine a 3.8 average weight for the iris ring variance. The UE 102 may multiple the average by 20 to arrive at value that may be used as a percentage match. In the instant example, 3.8 average weight would render a value of 76. In some example embodiments, the match probability may be a positive or negative match, e.g. match yes or no.

The UE may determine if the match probability satisfies a predetermined match threshold. The UE 102 may have one or more match thresholds based on the application or data which may be accessed. For example, general access to a UE 102 may only require a 70 percent match probability, whereas access to specific data, such as banking data, or business data may require a match probability of 90 percent. In an instance in which the match probability is The UE 102 may compare the match probability to the predetermined match threshold to determine if the predetermined match threshold is satisfied by the iris ring match probability. In an instance in which the predetermined match threshold is satisfied, the UE 102 may generate an indication of biometric data match or acceptance. The UE 102 may utilize the indication of biometric data match to access the secured devices, applications, or data. In an instance in which the predetermined match probability does not satisfy the match threshold, access to the secured information or device may continue to be restricted.

### Example Apparatus

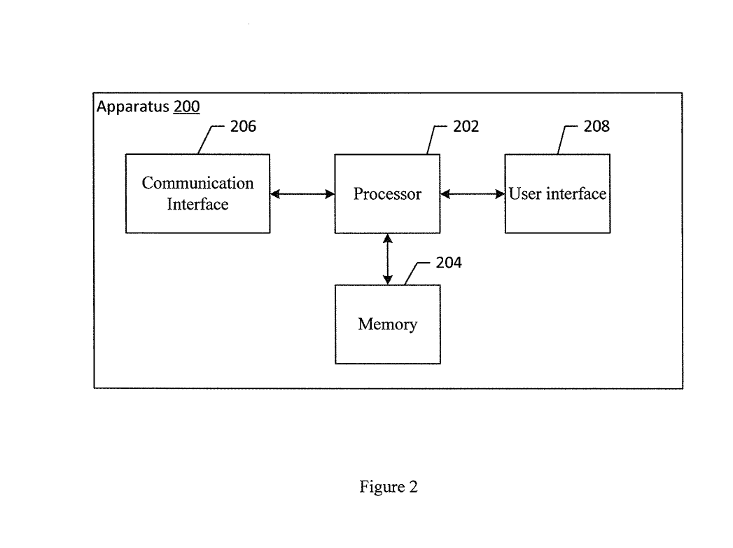

A UE 102 may include or otherwise be associated with an apparatus 200 as shown in FIG. 2. The apparatus, such as that shown in FIG. 2, is specifically configured in accordance with an example embodiment of the present invention to provide for iris recognition using natural light. The apparatus may include or otherwise be in communication with a processor 202, a memory device 204, a communication interface 206, and a user interface 208. In some embodiments, the processor (and/or co-processors or any other processing circuitry assisting or otherwise associated with the processor) may be in communication with the memory device via a bus for passing information among components of the apparatus. The memory device may be non-transitory and may include, for example, one or more volatile and/or non-volatile memories. In other words, for example, the memory device may be an electronic storage device (for example, a computer readable storage medium) comprising gates configured to store data (for example, bits) that may be retrievable by a machine (for example, a computing device like the processor). The memory device may be configured to store information, data, content, applications, instructions, or the like for enabling the apparatus to carry out various functions in accordance with an example embodiment of the present invention. For example, the memory device could be configured to buffer input data for processing by the processor. Additionally or alternatively, the memory device could be configured to store instructions for execution by the processor.

As noted above, the apparatus 200 may be embodied by UE 102. However, in some embodiments, the apparatus may be embodied as a chip or chip set. In other words, the apparatus may comprise one or more physical packages (for example, chips) including materials, components and/or wires on a structural assembly (for example, a baseboard). The structural assembly may provide physical strength, conservation of size, and/or limitation of electrical interaction for component circuitry included thereon. The apparatus may therefore, in some cases, be configured to implement an embodiment of the present invention on a single chip or as a single “system on a chip.” As such, in some cases, a chip or chipset may constitute means for performing one or more operations for providing the functionalities described herein.

The processor 202 may be embodied in a number of different ways. For example, the processor may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor may include one or more processing cores configured to perform independently. A multi-core processor may enable multiprocessing within a single physical package. Additionally or alternatively, the processor may include one or more processors configured in tandem via the bus to enable independent execution of instructions, pipelining and/or multithreading.

In an example embodiment, the processor 202 may be configured to execute instructions stored in the memory device 204 or otherwise accessible to the processor. Alternatively or additionally, the processor may be configured to execute hard coded functionality. As such, whether configured by hardware or software methods, or by a combination thereof, the processor may represent an entity (for example, physically embodied in circuitry) capable of performing operations according to an embodiment of the present invention while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (for example, a mobile terminal or a fixed computing device) configured to employ an embodiment of the present invention by further configuration of the processor by instructions for performing the algorithms and/or operations described herein. The processor may include, among other things, a clock, an arithmetic logic unit (ALU) and logic gates configured to support operation of the processor.

The apparatus 200 of an example embodiment may also include a communication interface 206 that may be any means such as a device or circuitry embodied in either hardware or a combination of hardware and software that is configured to receive and/or transmit data from/to a communications device in communication with the apparatus, such as to facilitate communications with one or more user equipment 110, utility device, or the like. In this regard, the communication interface may include, for example, an antenna (or multiple antennas) and supporting hardware and/or software for enabling communications with a wireless communication network. Additionally or alternatively, the communication interface may include the circuitry for interacting with the antenna(s) to cause transmission of signals via the antenna(s) or to handle receipt of signals received via the antenna(s). In some environments, the communication interface may alternatively or also support wired communication. As such, for example, the communication interface may include a communication modem and/or other hardware and/or software for supporting communication via cable, digital subscriber line (DSL), universal serial bus (USB) or other mechanisms.

The apparatus 200 may also include a user interface 208 that may, in turn, be in communication with the processor 202 to provide output to the user and, in some embodiments, to receive an indication of a user input. As such, the user interface may include a display and, in some embodiments, may also include a keyboard, a mouse, a joystick, a touch screen, touch areas, soft keys, one or more microphones, a plurality of speakers, or other input/output mechanisms. In one embodiment, the processor may comprise user interface circuitry configured to control at least some functions of one or more user interface elements such as a display and, in some embodiments, a plurality of speakers, a ringer, one or more microphones and/or the like. The processor and/or user interface circuitry comprising the processor may be configured to control one or more functions of one or more user interface elements through computer program instructions (for example, software and/or firmware) stored on a memory accessible to the processor (for example, memory device 204, and/or the like).

### Examples of Occlusions in Iris Image Iris Rings

FIG. 3 illustrates examples of occluded iris images. The left image depicts a reflection in the right section of the iris ring that occludes 46.0 percent of the iris ring. The enter image depicts a glare in the upper portion of the iris ring and a reflection in the right portion of the iris ring, occluding 63.8 percent of the iris ring. The right image depicts an reflection on the right and left portions of the iris ring which occludes 50.9 percent of the iris ring.

### Example Iris Images

FIGS. 4 and 5 illustrate example iris images. The iris image 400 may have an iris ring 408 defined by the pupil boundary 404 and the iris boundary 402. The pupil boundary 404 may be the area of the iris image 410 which encompasses the pupil with minimal or no inclusion of the iris area. The iris boundary 402 may the area in which the iris intersects the white of the eye, or limbus, which maximizes the iris area within the iris boundary and minimizes the white of the eye within the iris boundary. The iris ring 408 may have unclear regions 406. In FIG. 4, the iris ring 408 has two unclear regions, e.g. glare, one on either side of the pupil, near the pupil boundary 402. In FIG. 5, the iris ring 408 has an unclear region 406, e.g. a reflection, which extends from the pupil boundary 404 to the iris boundary 402 at the upper right portion of the iris ring.

### Example Iris Images Captured with Natural Light

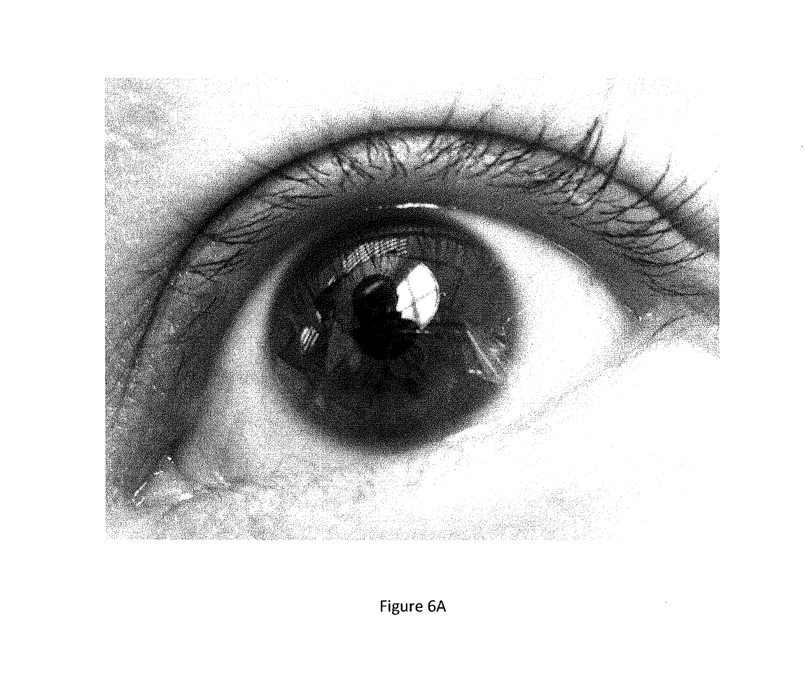

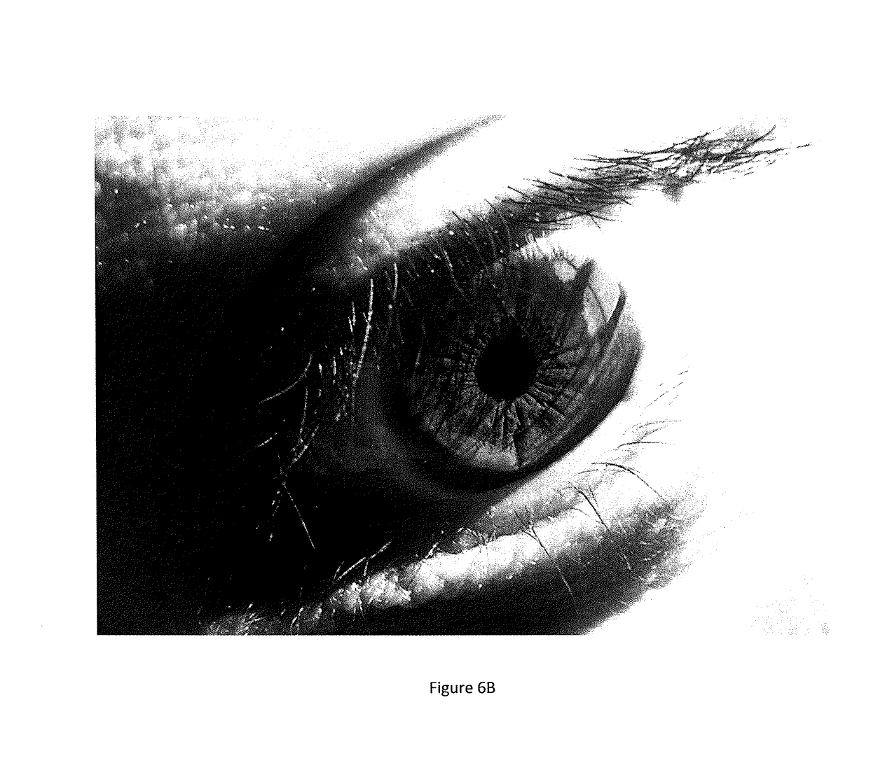

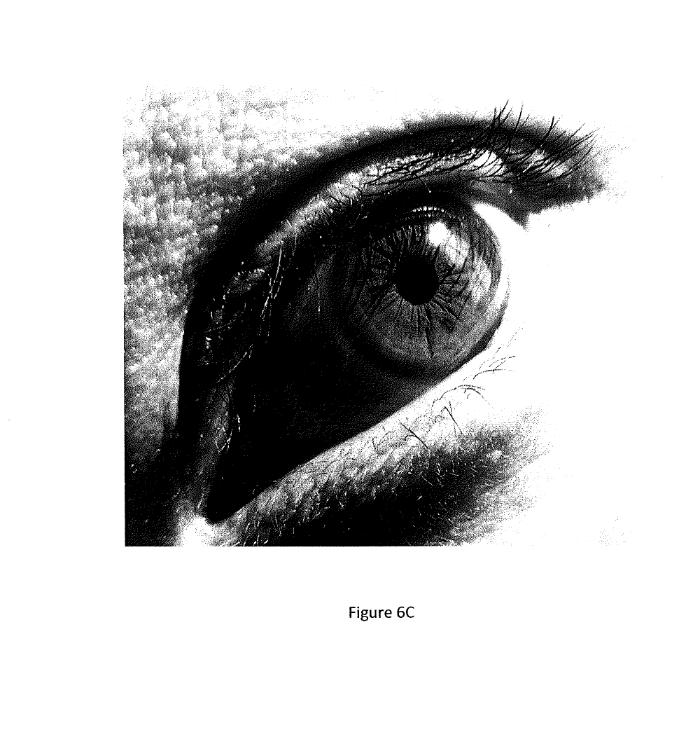

FIGS. 6A-6C illustrate example iris images captured using natural light. FIGS. 6A-6C depict iris image still photographs of an eye in three position captured using a mobile device camera using natural light. FIG. 6A is a depiction of the left eye positioned looking substantially straight ahead with the camera angled slightly below and eye and the light source in front of the eye. There is an reflection occlusion starting in the pupil region and upper portion of the iris ring in which the camera and the subject capturing the image are present. Additionally, there is a blur in the lower right portion of the iris ring. The iris ring of FIG. 6A has a clear lower quadrant.

FIG. 6B is a depiction of the eye looking substantially straight ahead with the camera slightly above the eye and the light source to the left of the eye. The iris ring has a occlusion, glare, in the upper right region and the lower half and left two thirds of the upper half of the iris ring are clear regions.

FIG. 6C is a depiction of the eye looking substantially straight ahead with the camera slightly to the right of the eye and the light source to the left of the eye. The iris ring has two occlusions. The first occlusion is a glare at the upper right area of the iris ring. The second occlusion is a reflection of a window in the right side of the iris ring. The lower, upper, and left quadrant of the iris ring are clear regions.

### Example Process for Iris Recognition Using Natural Light

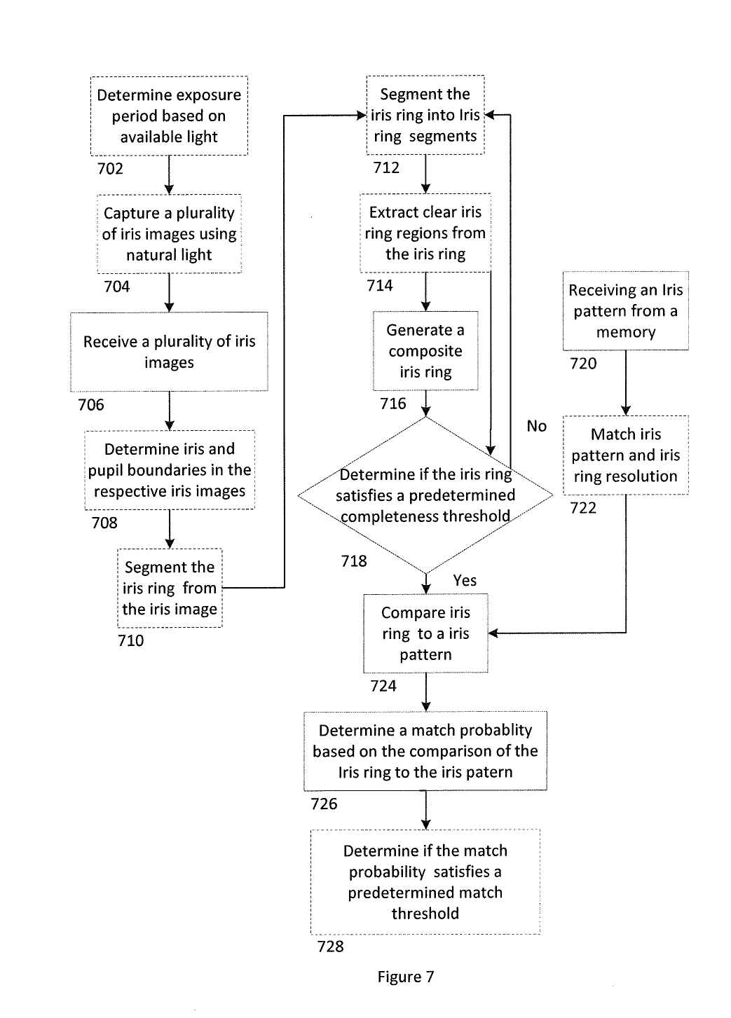

Referring now to FIG. 7, the operations performed, such as by the apparatus 200 of FIG. 2, for iris recognition using natural light are depicted. As shown in block 702 of FIG. 7, the apparatus 200 may include means, such as a processor 202, a communications interface 206, or the like, configured to determine an exposure time based on natural light. The communications interface 206 may request and/or receive light data from a photo sensor or light sensor associated with a camera 104. The processor 202 may receive the light data from the communications interface 206 and determine the exposure time for capturing one or more iris images 400 using natural light. Available natural light may include sun light or artificial light within the visible light range. Artificial light sources within the visible light range may include incandescent light sources, florescent light sources, light emitting diodes (LED), or the like. In some embodiments, the camera 104 may be equipped or associated with a light source that may be illuminated to increase the natural light available. The exposure time may be the time necessary for the camera 104 to capture an iris image using the available natural light.

As shown in block 704 of FIG. 7, the apparatus 200 may include a means, such as a processor 202, a communications module 206, or the like, configured to cause capture of a plurality of iris images using natural light. The processor 202 may cause the associated camera 104 to capture a plurality of iris images 400 based on the determined exposure time, using the communication interface 206. The camera 104 may be a portion of the apparatus 200, or be in association, by data communication, with the apparatus 200. The processor 202 may receive an indication of the eye 101 being positioned near the camera 104. The indication of eye 101 position may be automatically determined by the camera 104 and/or the processor 202 determining that an object, or specifically an eye 101, has been positioned near the camera aperture. Additionally or alternatively, the processor 202 may receive an indication of the eye 101 position based on a user input, such as selecting an “in position” or “ready” icon on a user interface 208, or the like.

The processor 202 may cause the camera 104 to capture one or more iris images using natural light in a first eye 101 position, such as straight ahead. The iris images may have occlusion areas, such as glare, reflections or blur, from natural light as depicted in FIG. 3. To compensate for occlusion areas, the processor 202 using the user interface 208 may prompt the user to reposition their eye 101, such as look left, right, up or down, reposition the camera in relationship to the light sources, or the like. The processor 202 may receive an indication of the user's eye 101 being in position and then cause one or more additional iris images to be captured by the camera 104. In some example embodiments the processor 202 may cause the camera 104 to capture three or more iris image positions.

In some example embodiments, the processor 202 may cause the iris images to be stored in local or remote temporary or long term storage, such as memory 204, for iris recognition processing.

As shown at block 706, of FIG. 7, the apparatus 200 may include means, such as a processor 202, memory 204, communications interface 206, or the like, configured to receive a plurality of iris images 400. The processor 204 may receive the plurality of iris images 400 from the memory 204 or the camera 104, using the communications interface 206.

As shown at block 708 of FIG. 7, the apparatus 200 may include means, such as a processor 202, or the like, configured to determine iris and pupil boundaries in respective iris images. As depicted in FIGS. 4 and 5, the processor 202 of an example embodiment may determine an iris boundary 402, such as a circular boundary at or near the intersection of the iris and the white of the eye, or limbus, which maximizes the iris area within the iris boundary and minimizes the white of the eye within the iris boundary. The processor 202 may also determine a pupil boundary 404 defining a pupil region, which may be the area of the iris image 400 which encompasses the pupil with minimal or no inclusion of the iris area. Additionally, the processor may determine unclear regions 408, such as glares, reflections, blurs, or the like, in which the iris pattern of the iris ring 408 is occluded.

As shown at block 710 of FIG. 7, the apparatus 200 may include means, such as a processor 202, or the like, configured to segment the iris ring from the iris image. The processor 202 may segment the iris ring 408 from each of the respective iris images 400 of the plurality of iris images. The processor 202 may remove the iris ring 408 from the iris image 400 and store the iris rings 408 in a temporary or long term memory, such as memory 204, for processing.

As shown at block 712 of FIG. 7, the apparatus 200 may include means, such as a processor 202, or the like, configured to segment the iris ring into iris ring segments. The processor 202 may further segment the iris rings into primary and secondary segments. For example, the full iris ring may be the primary segment and secondary segments may be portions of the iris ring, such as rows, columns, pie pieces, or the like, of the iris ring 408 for individual processing or composite composition.

As shown at block 714 of FIG. 7, the apparatus 200 may include means, such as a processor 202, or the like, configured to extract unclear iris ring regions from the iris rings. The processor 202 may remove unclear regions 406, e.g. glare, reflections, or blurs, from the iris rings 408. Additionally or alternatively, the processor 202 may remove secondary segments of the iris ring which contain unclear regions 406. After the removal of the unclear regions 406, clear iris ring regions or iris ring portions may remain for each iris ring 408.

In an alternative embodiment, the processor 202 may extract the clear portion of the iris ring 408 or clear iris segments from the iris ring for processing.

The process may continue at block 716 with the generation of a composite iris ring or at block 718 with the determination of the iris ring satisfying a completeness threshold.

As shown at block 716 of FIG. 7, the apparatus 200 may include means, such as a processor 202, or the like, configured to generate a composite iris ring. The processor 202 may identify the overlap of each iris ring 408 in relation to other iris rings in the plurality of iris rings, e.g. portions of the iris ring which are in multiple iris images 400. For example, using the iris images of FIG. 3, a full iris ring is present, although not completely clear in all three iris images. The processor 202 may select the iris ring 408 with the most clear iris segments or the most complete iris ring after the extraction of the unclear regions 406. Continuing the example, the processor 202 may select the left iris image with 46.0 percent occlusion, as 54.0 percent will be clear and will be the most complete iris ring. The processor 202 may fill in the missing segments or iris ring regions using clear segments or regions from the other iris image 400 iris rings 408 from the plurality of iris images 400 to generate a composite iris ring in which the whole iris ring is clear, or the maximum available clear iris ring is represented. The processor 202 weight the remaining iris images for preference in selecting clear segments or regions for iris ring fill in. Returning to the example, portions of the center and right image may be used to fill in the missing regions or segments of the left iris image iris ring. The right image may be weighted as preferred and all available clear portions or segments may be used before clear portions of the center iris image are used to fill in the missing portions of the iris ring.

In an example embodiment, the processor 202 may generate composite iris ring by averaging the clear iris ring region or segments. The clear iris ring segments may be aligned, e.g. portions of each iris image iris ring may be matched with portions of other iris image iris rings and associated. The processor 202 may then average the clear iris ring segments or regions, such as by using Gaussian Mixed Models (GMM), or other image averaging model.

A shown at block 718 of FIG. 7, the apparatus 200 may include means, such as a processor 202, or the like, configured to determine if the iris ring satisfies a predetermined iris completeness threshold. The processor 202 may compare the iris ring completeness to a completeness threshold, such as 80, 85, 90, 95 or any other percentage of completeness. In an instance in which the completeness threshold is satisfied the process may continue at block 624 by comparing the iris ring to the iris pattern. In an instance in which the completeness threshold is not satisfied, the process may recommence at block 602 by determining an exposure period based on available light. The exposure period may be lengthened to increase the image clarity while using less or the same amount of light, then the process would be repeated capturing and processing iris images with the new exposure period.

As shown at block 720 of FIG. 7, the apparatus 200 may include means, such as a processor 202, a memory 204, a communications interface 206, or the like, configured to receive an iris pattern from memory. The processor 202 may request an iris pattern from a memory 204, such as an iris pattern database 106. The iris pattern database 106 may be a local or remote database storing one or more iris patterns. The processor 202 may receive the iris pattern from the memory 204. In embodiments in which the memory is not local to the device, the processor 202 may use the communications interface 206 for data communication with the memory 204.

As shown at block 722 of FIG. 7, the apparatus 200 may include means, such as a processor 202, or the like, configured to match iris image and iris pattern resolution. The processor 202 may adjust the resolution by increasing or decreasing the area per pixel resolution of the iris pattern, to match the area per pixel resolution of the composite iris ring or most complete iris ring.

As shown in block 724 of FIG. 7, the apparatus 200 may include means, such as a processor 202, or the like, configured to compare the iris ring to the iris pattern. The processor 202 may compare the most complete iris ring 408 or the composite iris ring to the iris pattern received from memory. The processor 202 may identify iris pattern identification points associated with the iris ring, such as rings, furrows, freckles, or the like, and compare the iris ring identification points to the identification points of the iris pattern.

Additionally or alternatively, the UE 102 may determine a match be determining a variance, e.g. uncertainty, for each area of the iris region. An area of the iris region may be the entire iris ring iris ring subsections, iris ring segments, individual pixels, or any other sub division of the iris ring. The UE 102 may assign a variance weight to each iris ring area, the weight may be scaled or binary, such as a 1-5, 5 being no variation and 1 being highest variation, or in binary 1 being no or little variation, e.g. match and 0 being variation satisfying a variation threshold, e.g. no match. The UE 102 may determine the average weight of the iris ring areas.

As shown at block 726 of FIG. 7, the apparatus 200 may include means, such as a processor 202, or the like, configured to determine a match probability based on the comparison of the iris ring to the iris pattern. The processor 202 may determine a match probability based on the number of identification points which are determined to be a match based on the comparison of the iris ring to the iris pattern and/or the average weight of the iris ring variance. A match probability may be a percentage of matching identification points or a function of the average weight of the iris variance. For example, in an instance in which the variation is scaled 1-5, 5 being no variation and 1 being highest variation, the UE may determine a 3.8 average weight for the iris ring variance. The UE 102 may multiple the average by 20 to arrive at value that may be used as a percentage match. In the instant example, 3.8 average weight would render a value of 76. In an example embodiment, the match probability may be a positive or negative match, e.g. match yes or no.

As shown at block 728 of FIG. 7, the apparatus 200 may include means, such as a processor 202, or the like, configured to determine if the match probability satisfies a predetermined match threshold. The processor 202 may have one or more match thresholds based on the application or data which may be accessed. For example, general access to a UE 102 may only require a 70 percent match probability, whereas access to specific data, such as banking data, or business data may require a match probability of 90 percent. In an instance in which the match probability is a positive or negative match, the match threshold may be positive match. The processor 202 may compare the match probability to the match threshold to determine if the predetermined match threshold is satisfied by the iris ring match probability. In an instance in which the match threshold is satisfied, the processor 202 may generate an indication of biometric data match or acceptance. The processor 202 may utilize the indication of biometric data match to access the secured devices, applications, or data. In an instance in which the match probability does not satisfy the predetermined match threshold, access to the secured information or device may continue to be restricted.

The implementation of a iris recognition using natural light may allow iris recognition biometric security to be utilized by mobile devices or in some instances stationary devices. Determining an exposure time may allow the cameras which are installed, as standard, on mobile devices and wearable computing devices to be used, instead of requiring a separate camera specially configured for iris recognition to be used.

The capture of a plurality of images allows for the extraction of unclear regions of the iris rings and comparison of the most complete iris ring or a composite iris ring, thus removing the drawback of occlusions created by the use of natural light.

As described above, FIG. 7 illustrates a flowchart of an apparatus 200, method, and computer program product according to example embodiments of the invention. It will be understood that each block of the flowchart, and combinations of blocks in the flowchart, may be implemented by various means, such as hardware, firmware, processor, circuitry, and/or other communication devices associated with execution of software including one or more computer program instructions. For example, one or more of the procedures described above may be embodied by computer program instructions. In this regard, the computer program instructions which embody the procedures described above may be stored by a memory device 204 of an apparatus employing an embodiment of the present invention and executed by a processor 202 of the apparatus. As will be appreciated, any such computer program instructions may be loaded onto a computer or other programmable apparatus (for example, hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowchart support combinations of means for performing the specified functions and combinations of operations for performing the specified functions for performing the specified functions. It will also be understood that one or more blocks of the flowchart, and combinations of blocks in the flowchart, can be implemented by special purpose hardware-based computer systems which perform the specified functions, or combinations of special purpose hardware and computer instructions.

In some embodiments, certain ones of the operations above may be modified or further amplified. Furthermore, in some embodiments, additional optional operations may be included, such as illustrated by the dashed outline of block 702, 704, 708, 710, 714, and 726 in FIG. 7. Modifications, additions, or amplifications to the operations above may be performed in any order and in any combination.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

That which is claimed:

1. A method comprising:
receiving, at a user device, a plurality of iris images, wherein the iris images are captured using natural light;
generating a composite iris ring based on the plurality of iris images;
receiving an iris image pattern;
comparing the composite iris ring to the iris pattern; and
determining a match probability based on the comparison of the composite iris ring to the iris pattern.

2. The method of claim 1 further comprising:
determining an iris boundary and a pupil boundary defining an iris ring in the respective iris images of the plurality of iris images; and
segmenting the iris ring from the respective iris images of the plurality of iris images.

3. The method of claim 2 further comprising:
extracting unclear regions from the iris ring, wherein the generating a composite iris ring is further based on clear regions of the iris ring.

4. The method of claim 1 further comprising:
matching iris pattern and iris ring resolution, wherein the comparison of the iris ring and iris pattern is further based on the iris ring and iris pattern having a matched resolution.

5. The method of claim 1 further comprising:
determining an exposure period based on available natural light; and
capturing a plurality of iris images, wherein the capture of the plurality of iris images is performed using the determined exposure period.

6. The method of claim 1 further comprising:
determining if the match probability satisfies a predetermined match threshold.

7. The method of claim 2 further comprising:
segmenting the iris ring into iris ring segments.

8. An apparatus comprising at least one processor and at least one memory including computer program code, the at least one memory and computer program code configured to, with the processor, cause the apparatus to at least:
receive a plurality of iris images, wherein the iris images are captured using natural light;
generate a composite iris ring based on the plurality of iris images;
receive an iris image pattern;
compare the composite iris ring to the iris pattern; and
determine a match probability based on the comparison of the composite iris ring to the iris pattern.

9. The apparatus of claim 8, wherein the at least one memory and the computer program code are further configured to:
determine an iris boundary and a pupil boundary defining an iris ring in the respective iris images of the plurality of iris images; and
segment the iris ring from the respective iris images of the plurality of iris images.

10. The apparatus of claim 9, wherein the at least one memory and the computer program code are further configured to:
extract unclear regions from the iris ring, wherein the generating a composite iris ring is further based on clear regions of the iris ring.

11. The apparatus of claim 8, wherein the at least one memory and the computer program code are further configured to:
match iris pattern and iris ring resolution, wherein the comparison of the iris ring and iris pattern is further based on the iris ring and iris pattern having a matched resolution.

12. The apparatus of claim 8, wherein the at least one memory and the computer program code are further configured to:
determine an exposure period based on available natural light; and
capture a plurality of iris images, wherein the capture of the plurality of iris images is performed using the determined exposure period.

13. The apparatus of claim 8, wherein the at least one memory and the computer program code are further configured to:
determine if the match probability satisfies a predetermined match threshold.

14. The apparatus of claim 8, wherein the at least one memory and the computer program code are further configured to:
segment the iris ring into iris ring segments.

15. A computer program product comprising at least one non-transitory computer-readable storage medium having computer-executable program portions stored therein, the computer-executable program code portions comprising program code instructions configured to:
receive a plurality of iris images, wherein the iris images are captured using natural light;
generate a composite iris ring based on the plurality of iris images;
receive an iris image pattern;
compare the composite iris ring to the iris pattern; and
determine a match probability based on the comparison of the composite iris ring to the iris pattern.

16. The computer program product of claim 15, wherein the computer-executable program code portions further comprise program code instructions configured to:
determine an iris boundary and a pupil boundary defining an iris ring in the respective iris images of the plurality of iris images; and
segment the iris ring from the respective iris images of the plurality of iris images.

17. The computer program product of claim 15, wherein the computer-executable program code portions further comprise program code instructions configured to:
segment the iris ring into iris ring segments;
extract unclear regions from the iris ring, wherein the unclear regions are unclear iris ring segments; and
wherein the generating a composite iris ring is further based on clear regions of the iris ring.

18. The computer program product of claim 15, wherein the computer-executable program code portions further comprise program code instructions configured to:
match iris pattern and iris ring resolution, wherein the comparison of the iris ring and iris pattern is further based on the iris ring and iris pattern having a matched resolution.

19. The computer program product of claim 15, wherein the computer-executable program code portions further comprise program code instructions configured to:
determine an exposure period based on available natural light; and
capture a plurality of iris images, wherein the capture of the plurality of iris images is performed using the determined exposure period.

20. The computer program product of claim 15, wherein the computer-executable program code portions further comprise program code instructions configured to:
determine if the match probability satisfies a predetermined match threshold.
